(19) United States
(12) Patent Application Publication
(10) Pub. No.: US 2011/0027358 A1
(43) Pub. Date: Feb. 3, 2011

(54) VALSARTAN TABLET FORMULATIONS

(76) Inventors: Rajesh Kshirsagar, Gujarat (IN); Sachin Mundade, Gujarat (IN); Dipti Ranjan Parida, Gujarat (IN)

Correspondence Address: MERCHANT & GOULD PC, P.O. BOX 2903, MINNEAPOLIS, MN, 55402-0903, US

(21) Appl. No.: 12/532,535

(22) PCT Filed: Mar. 31, 2008
(86) PCT No.: PCT/IN2008/000208

§ 371 Date: Sep. 27, 2010

(30) Foreign Application Priority Data
Mar. 29, 2007 (IN) 1609/MUM/2006

(51) Int. Cl. A61K 31/41; A61K 9/28

(52) U.S. Cl. 424/465; 424/474; 514/381

(57) ABSTRACT

The present invention relates to a pharmaceutical tablet composition comprising an effective amount of valsartan. The tablet is prepared by wet granulation and exhibits satisfactory disintegration properties. The invention also relates to a process for preparation of a pharmaceutical tablet composition comprising an effective amount of valsartan wherein the process involves a wet granulation step.

### FIELD OF THE INVENTION

The present invention relates to solid oral dosage forms of valsartan. In particular, the present invention relates to pharmaceutical tablet compositions comprising an effective amount of valsartan.

### BACKGROUND OF THE INVENTION

Valsartan is an orally active angiotensin II antagonist acting on the AT₁, receptor subtype and is prescribed for the treatment of hypertension and heart failure. Chemically it is (S)—N-(1-Carboxy-2-methyl-prop-1-yl)-N-pentanoyl-N-[2′-(1H-tetrazol-5-yl)-bi phenyl-4-ylmethyl]-amine. Valsartan is marketed as tablets intended for oral administration under the trade name DIOVAN® (Novartis) in strengths of 40 mg, 80 mg, 160 mg and 320 mg of valsartan.

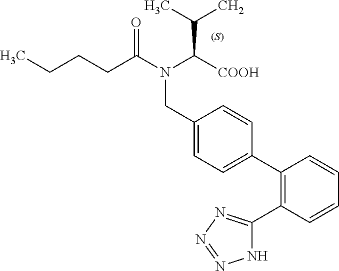

U.S. Pat. No. 5,399,578 describes the preparation of valsartan and its pharmaceutically acceptable salts. U.S. Pat. No. 6,294,197, U.S. Pat. No. 6,485,745 and U.S. Pat. No. 6,858,228 describe a solid oral dosage form of valsartan and optionally hydrochlorothiazide (HCTZ). These patents disclose that valsartan is difficult to formulate and therefore it has not been possible to make oral formulations in the form of tablets in a reliable and robust way. The patents further suggest the preparation of compressed tablets of valsartan by a dry granulation (slugging) technique. However slugging requires specialized equipment and is often time consuming. It also involves critical steps like roll compaction, screening and recompaction. This causes a considerable loss of the material and thereby results in poor yield of the final product. The criticalities of the steps also mean that the process can be variable.

WO 2005/089720 states that valsartan tablets when formulated have disintegration problems as valsartan, being a fluffy material, when compressed it leads to the formation of a high-density product which is problematic in that it does not disintegrate satisfactorily, which leads to improper dissolution and sub-therapeutic concentration levels. The application further suggests valsartan tablets for oral administration comprising valsartan, at least two different disintegrants, and optionally hydrochlorthiazide (HCTZ).

Thus there remains an unmet need for a simple and robust process to prepare valsartan tablets that exhibit satisfactory disintegration behavior.

We have now surprisingly found that it is possible to prepare tablets comprising valsartan by a simple and economic wet granulation method, wherein the tablets exhibit satisfactory disintegration properties.

### OBJECT OF THE INVENTION

An object of the present invention is to provide a pharmaceutical tablet composition comprising an effective amount of valsartan wherein the tablet is prepared by wet granulation and wherein the tablet has satisfactory disintegration properties

Yet another object of the present invention is to provide a process for the preparation of a pharmaceutical tablet composition comprising an effective amount of valsartan wherein the process involves a wet granulation step.

### SUMMARY OF THE INVENTION

According to one aspect of the present invention there is provided a pharmaceutical tablet composition comprising effective amount of valsartan and binder and having satisfactory disintegration properties wherein, the tablet is prepared by wet granulation and wherein the binder is pregelatinized starch.

According to yet another aspect of the present invention there is provided a process for the preparation of a pharmaceutical tablet composition comprising an effective amount of valsartan wherein the process involves a wet granulation step.

### DETAILED DESCRIPTION OF THE INVENTION

The present invention relates to a pharmaceutical tablet composition comprising an effective amount of valsartan wherein the tablet is prepared by wet granulation.

By “effective amount”, it is meant that amount of active agent, which halts or reduces the progress of the condition being treated or which otherwise completely or partly cures or acts palliatively on the condition. In a preferred embodiment, the effective amount of valsartan can be from 10-320 mg for example 40, 80, 160 or 320 mg.

The tablet composition exhibits satisfactory disintegration properties. By “satisfactory” it is meant that disintegration behaviour which provides satisfactory dissolution and therefore therapeutic concentration in the blood.

The tablet composition may further comprise pharmaceutically acceptable excipients known in the art which can, for example, provide bulk and aid in processing. These include but are not limited to disintegrants, binders, fillers or diluents, lubricants, glidants, surfactants and the like.

The tablets of the present invention, comprise of croscarmellose sodium as the disintegrant. The concentration of disintegrant may vary from about 1% to about 20%, more preferably from about 5% to about 15% by weight of the tablet.

The tablets of the present invention comprise of pregelatinized starch as the binder. The concentration of binder may vary from about 0.1% to about 10%, more preferably from about 0.5% to about 5% by weight of the tablet.

In a preferred embodiment, the tablet composition of the invention comprises of croscarmellose sodium as the disintegrant in a concentration from about 5% to about 15% by weight of the tablet and pregelatinized starch as the binder in a concentration from about 0.5% to about 5% by weight of the tablet.

Examples of fillers or diluents include but are not limited to calcium salts such as calcium carbonate, calcium phosphate-dibasic, calcium phosphate-tribasic, calcium sulfate and the like; cellulose derivatives such as microcrystalline cellulose, silicified microcrystalline cellulose and the like and saccharides such as lactose, starch, mannitol and the like. In a preferred embodiment, the diluent used is a combination of lactose monohydrate and microcrystalline cellulose.

Suitable lubricants include stearic acid and stearates, canola oil, glyceryl palmitostearate, hydrogenated vegetable oil, mineral oil, polyethylene glycols, sodium stearyl fumarate, talc and the like. In a preferred embodiment, magnesium stearate is included as a lubricant in an amount from about 0.5% to about 1.5% by weight of the tablet.

Suitable glidants include colloidal silicon dioxide, magnesium trisilicate and the like. In a preferred embodiment, colloidal silicon dioxide is included as a glidant in an amount up to about 2%, preferably from about 0.5% to about 1.5%, by weight of the tablet.

Examples of surfactants include, but are not limited to poloxamers, sodium lauryl sulphate, polysorbates and the like. In a preferred embodiment, poloxamer (for example marketed under the trade name Lutrrol® F 68) is included as a surfactant in an amount up to about 3%, preferably from about 0.1% to about 1.0%, by weight of the tablet.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention.

In another aspect, the invention provides a process of preparation of a pharmaceutical tablet composition comprising an effective amount of valsartan as hereinabove described comprising the steps of:

i) Sifting the accurately weighed quantities of active agent and one or more pharmaceutically acceptable additives through a suitable sieve followed by mixing.
ii) Granulating the mix of step (i) with an aqueous solution of a surfactant.
iii) Drying the granulated mass at room temperature and sifting through a suitable sieve
iv) Prelubricating the sifted blend of step (iii) with sifted extragranular excipients followed by lubrication with sifted lubricant(s) and
v) Compressing the lubricated granules into tablets

The granulation can be performed using any of the conventional equipments well known to the person skilled in the art. In a preferred embodiment, a rapid mixer granulator is used for granulation.

The valsartan tablets may further be coated with one or more non-functional layers comprising film-forming polymers and optionally one or more other coating additives, if desired. The tablets can be coated by using any of the conventional coating techniques and utilizing conventional equipments well known to the persons skilled in the art. The one or more coatings may be applied from aqueous or non-aqueous systems or combinations selected from the group comprising thereof as appropriate. The solvent used in the non-aqueous coating comprises isopropyl alcohol, acetone, methanol, dichloromethane and mixtures thereof. The non-functional coating layers comprise of one or more excipients selected from the group consisting of film forming agents, adhesion promoting agents, plasticizers, opacifiers, colouring agents, antitacking agents and the like.

Examples of film forming polymers include polysaccharides such as maltodextrin; alkyl celluloses such as methyl or ethyl cellulose, hydroxyalkylcelluloses (e.g. hydroxypropylcellulose or hydroxypropylmethylcelluloses); polyvinylpyrrolidone, polyvinyl alcohol, copolymers of vinylpyrrolidone and vinyl acetate (e.g. marketed under the brand name of Plasdone®) polymers based on methacrylic acid such as those marketed under the brand name of Eudragit®, alginates and the like.

Examples of adhesion promoting agents in film coating include, but are not limited to lactose, microcrystalline cellulose and the like. Plasticizers are selected from the group comprising, but are not limited to, dibutyl phthalate, triethyl citrate, polyethylene glycol, surfactants such as polysorbates and the like and mixtures thereof. A suitable opacifier is titanium dioxide. Coloring agents may be selected from, but are not limited to, those conventionally known in the art such as iron oxide red, sunset yellow, black iron oxide, yellow iron oxide and the like. Antitacking agents include talc, stearic acid its salts and derivatives, and colloidal silicon dioxide and the like.

In a preferred embodiment, the commercially available coating composition Opadry® is used as a coating agent.

The following are few representative examples of the invention and in no way construed as limiting the invention.

TABLE 1
Composition of valsartan tablets of Example 1 to 4

| | % w/w of tablet | | | |
|---|---|---|---|---|
| Ingredients | Example 1 | Example 2 | Example 3 | Example 4 |
| Valsartan | 25.06 | 51.61 | 53.33 | 35.71 |
| Microcrystalline | 23.96 | 14.52 | — | — |
| cellulose | | | | |
| (Avicel ® PH 101) | | | | |
| Lactose monohydrate | 35.94 | 19.60 | 20.83 | 35.71 |
| (Granulac ® 200) | | | | |
| Lactose monohydrate | — | — | 21.09 | — |
| (Flowlac ® 200) | | | | |
| Sodium starch | — | — | 4.00 | — |
| glycolate (Glycolis ®) | | | | |
| Corn starch | — | — | — | 25.00 |
| Purified talc | — | — | — | 3.04 |
| Povidone K-30 | — | 4.83 | — | — |
| (Plasdone ® K 29/32) | | | | |
| Croscarmellose sodium | — | 4.84 | — | — |
| Crospovidone | 6.79 | — | — | — |
| (Polyplasdone ®) | | | | |
| Poloxamer | — | 2.90 | — | — |
| Colloidal silicon | 1.46 | 0.73 | — | — |
| dioxide (Aerosil ®-200) | | | | |
| Crospovidone | 2.91 | — | — | — |
| (Polyplasdone ® XL) | | | | |
| Magnesium stearate | 0.97 | 0.97 | 0.75 | 0.54 |
| Opadry ® 02F50107 | 2.91 | — | — | — |
| purple | | | | |
| Total | 100.00 | 100.00 | 100.00 | 100.00 |

### Brief Manufacturing Process

### Example 1

Valsartan, microcrystalline cellulose, lactose monohydrate, and crospovidone were mixed together. This mixture was then granulated with purified water, allowed to dry and then prelubricated with crospovidone and colloidal silicon dioxide and lubricated with magnesium stearate. The lubricated granules were compressed into tablets. The tablets were then coated using aqueous Opadry®.

### Example 2

Valsartan, lactose monohydrate, microcrystalline cellulose and croscarmellose sodium were mixed together. This mixture was then granulated with poloxamer solution, allowed to dry, prelubricated with croscarmellose sodium and colloidal silicon dioxide and lubricated with magnesium stearate. The lubricated granules were then compressed into tablets.

### Example 3

Valsartan, lactose monohydrate, and sodium starch glycolate were mixed together. This mixture was then granulated with water, allowed to dry, prelubricated with Sodium starch glycolate and lubricated with magnesium stearate. The lubricated granules were then compressed into tablets.

### Example 4

Valsartan & lactose monohydrate were mixed together. This mixture was then granulated with starch paste, allowed to dry, prelubricated with starch & talc and lubricated with magnesium stearate. The lubricated granules were then compressed into tablets.

TABLE 2
Composition of valsartan tablets of Example 5 to 7

| | % w/w of tablet | | |
|---|---|---|---|
| | Example 5 | Example 6 | Example 7 |
| Ingredients | | | |
| Valsartan | 25.06 | 50.11 | 51.78 |
| Microcrystalline cellulose | 34.31 | 6.26 | 6.47 |
| (Avicel ® PH101) | | | |
| Lactose monohydrate | 15.86 | 24.44 | 19.56 |
| (Granulac ® 200) | | | |
| Pregelatinized starch | 2.44 | 2.43 | 0.97 |
| (Starch ® 1500 LM) | | | |
| Croscarmellose sodium | 4.71 | 1.94 | 4.86 |
| (Ac-di-sol ®) | | | |
| Poloxamer | 0.49 | 0.49 | 0.48 |
| (Lutrol ® F 68) | | | |
| Extragranular excipients | | | |
| Lactose monohydrate | 7.85 | 7.83 | 6.41 |
| (Granulac ® 200) | | | |
| Croscamellose sodium (Ac-di-sol ®) | 4.71 | 1.94 | 4.86 |
| Colloidal silicon dioxide, anhydrous | 0.72 | 0.70 | 0.73 |
| Magnesium stearate | 0.94 | 0.95 | 0.97 |
| Opadry ® | 2.91 | 2.91 | 2.91 |
| Total | 100.00 | 100.00 | 100.00 |

### Brief Manufacturing Process

### Examples 5-7

Valsartan, microcrystalline cellulose, lactose monohydrate, pregelatinized starch and croscaramellose sodium were mixed together. This mixture was then granulated with poloxamer solution, allowed to dry and then prelubricated with lactose monohydrate, croscarmellose sodium and colloidal silicon dioxide and lubricated with magnesium stearate. The lubricated granules were compressed into tablets. The tablets were then coated using aqueous Opadry®.

TABLE 3
Disintegration time of Examples 1-7

| | Disintegration Time | |
|---|---|---|
| Example | in Minutes | Surface texture after coating |
| Innovator | 1-2 | Smooth |
| Example 1 | 2 | Rough |
| Example 2 | ≧15 | — |
| Example 3 | 8-10 | — |
| Example 4 | ≧15 | — |
| Example 5 | 2-3 | Smooth |
| Example 6 | 3-4 | Smooth |
| Example 7 | 3-4 | Smooth |

The tablets of all the examples were evaluated for their disintegration time and surface texture after coating where applicable. The disintegration time was evaluated after performing the disintegration test as per USP 31, vol. 1, pp 266. Binders like starch paste (Example 4) and Povidone K-30 (Example 2) and disintegrants like sodium starch glycolate (Example 3) led to very high disintegration time for tablets. Though disintegrants like crospovidone (Example 1) gave satisfactory disintegration time, but the tablets containing crospovidone were observed to have rough surface after coating. Only pregelatinized starch as a binder and croscaramellose sodium as disintegrant were found to exhibit a synergistic effect to give tablets with satisfactory disintegration time and acceptable surface texture after coating (Examples 5 to 7).

### Dissolution Method

The tablets of examples 5 to 7 were tested for dissolution of valsartan in 1000 ml of phosphate buffer of pH 6.8 as dissolution medium at 37° C. in USP Type II apparatus, rotated at 50 rpm. The dissolution data obtained was tabulated and compared with that of the innovator.

TABLE 4
In vitro dissolution data

| Time(min) | Innovator | Example 5 | Example 6 | Example 7 |
|---|---|---|---|---|
| 5 | 86 | 61 | 79 | 82 |
| 10 | 100 | 99 | 96 | 101 |
| 15 | 101 | 101 | 98 | 103 |
| 20 | 101 | 102 | 99 | 104 |
| 30 | 101 | 102 | 99 | 104 |
| 45 | 101 | 102 | 99 | 104 |
| 60 | 101 | 102 | 99 | 104 |

The dissolution data obtained clearly shows that valsartan tablets formulated with wet granulation technique matched with that of the innovator. This indicates that valsartan tablets can be prepared using a wet granulation method reliably.

1. Pharmaceutical tablet composition comprising an effective amount of valsartan and binder and having satisfactory disintegration properties wherein, the tablet is prepared by wet granulation and wherein the binder is pregelatinized starch.

2. A tablet composition according to claim 1, wherein valsartan is present in a unit dose from 40 mg to 320 mg.

3. A tablet composition according to claim 1, wherein pharmaceutically acceptable additives are selected from the group comprising fillers or diluents, lubricants, glidants and disintegrants.

4. A tablet composition according to claim 3 wherein the disintegrant is croscarmellose sodium.

5. A tablet composition according to claim 1, wherein the disintegrant is present in an amount from about 1 to 20% by weight of the tablet.

6. A tablet composition according to claim 1, wherein the pregelatinized starch is present in an amount from about 0.1% to 10% by weight of the tablet.

7. A tablet composition according to claim 1, wherein the pregelatinized starch is present in an amount from about 0.5% to 5% by weight of the tablet.

8. A tablet composition according to claim 3, wherein the diluent is a mixture of lactose monohydrate and microcrystalline cellulose

9. A tablet composition according to claim 3, wherein the lubricant is magnesium stearate.

10. A tablet composition according to claim 3, wherein the glidant is colloidal silicon dioxide.

11. A tablet composition according to claim 1, wherein the tablet is further coated with one or more coating layers comprising film forming agents, adhesion promoting agents, coating agents, plasticizers, antitacking agents, coloring agents, opacifiers or mixtures thereof.

12. A pharmaceutical tablet composition prepared by a process involving a wet granulation step having satisfactory disintegration properties comprising:
a) Valsartan
b) Pregelatinized starch
c) Croscarmellose sodium and one or more pharmaceutically acceptable additives.

13. A process of preparation of a pharmaceutical tablet composition comprising effective amount of valsartan which has satisfactory disintegration properties, the process comprising:
i) sifting the accurately weighed quantities of valsartan, pregelatinized starch as binder, and one or more pharmaceutically acceptable additives through a suitable sieve followed by mixing.
ii) granulating the mix of step (i) with aqueous solution of a surfactant.
iii) drying the granulated mass at room temperature and sifting through a suitable sieve.
iv) prelubricating the sifted blend of step (iii) with sifted extragranular material followed by lubrication with sifted lubricant(s) and
v) compressing the lubricated granules into tablets.

14. The process of claim 13 wherein the surfactant is poloxamer.
